(19) United States
(12) Patent Application Publication
(10) Pub. No.: US 2024/0120057 A1
(43) Pub. Date: Apr. 11, 2024

(54) Artificial Intelligence For Determining A Patient's Disease Progression Level to Generate A Treatment Plan

(71) Applicant: HEALTHPOINTE SOLUTIONS, INC., Austin, TX (US)

(72) Inventors: Nathan Gnanasambandam, Irvine, CA (US); Mark Henry ANDERSON, Newport Coast, CA (US)

(21) Appl. No.: 18/553,314

(22) PCT Filed: Mar. 31, 2022
(86) PCT No.: PCT/US2022/022919

§ 371 Date: Sep. 29, 2023

Related U.S. Application Data
(63) Continuation-in-part of application No. 17/674,604, filed on Feb. 17, 2022.
(60) Provisional application No. 63/168,855, filed on Mar. 31, 2021.

(51) Int. Cl. G16H 20/10; G16H 15/00; G16H 50/20; G16H 50/30; G16H 50/70

(52) U.S. Cl. CPC G16H 20/10; G16H 15/00; G16H 50/20; G16H 50/30; G16H 50/70

(57) ABSTRACT

Systems, methods, and computer-readable mediums for generating, by an artificial intelligence engine, a treatment plan for a medical condition of a patient. The method comprises receiving medical data pertaining to the patient. The method also comprises determining, by the artificial intelligence engine and by using machine learning models, a disease progression level for the medical condition of the patient. The disease progression level indicates a risk of the patient reaching a next stage on a disease continuum of the medical condition. The method further comprises generating, by the artificial intelligence engine, the treatment plan for the medical condition. The generating is based at least on the disease progression level. The treatment plan comprises one or more actionable items to be performed on or by the patient. The method also comprises transmitting the treatment plan to a computing device for presentation to a healthcare professional.

### CROSS-REFERENCES TO RELATED APPLICATIONS

This application claims priority to U.S. Provisional Application No. 63/168,855 filed Mar. 31, 2021, and is a CIP (Continuation in part) of U.S. patent application Ser. No. 17/674,604, filed Feb. 17, 2022. All applications are hereby incorporated by reference in their entirety for all purposes as if reproduced in full below.

### BACKGROUND

Population health management entails aggregating patient data across multiple health information technology resources, analyzing the data with reference to a single patient, and generating actionable items through which care providers can improve both clinical and financial outcomes. A population health management service seeks to improve the health outcomes of a group by improving clinical outcomes while lowering costs.

### SUMMARY

Care pathway may provide specific sets of evidence-based recommendations tailored to treat each stage of a medical condition. Further tailoring evidence-based recommendations to account for a disease progression level of the medical condition may improve patient outcome. Accordingly, the present disclosure provides systems, methods, and non-transitory computer-readable media for, among other things, generating a treatment plan for a medical condition of a patient based on a disease progression level.

The present disclosure provides a method for generating, by an artificial intelligence engine, a treatment plan for a medical condition of a patient. The method comprises receiving medical data pertaining to the patient. The method also comprises determining, by the artificial intelligence engine and by using one or more machine learning models, a disease progression level for the medical condition of the patient. The determining is based at least on the medical data. The disease progression level indicates a risk of the patient reaching a next stage on a disease continuum of the medical condition. The method further comprises generating, by the artificial intelligence engine, the treatment plan for the medical condition. The generating is based at least on the disease progression level. The treatment plan comprises one or more actionable items to be performed on or by the patient. The method also comprises transmitting the treatment plan to a computing device for presentation to a healthcare professional.

The present disclosure also provides a system for generating, by an artificial intelligence engine, a treatment plan for a medical condition of a patient. The system comprises, in one implementation, a memory device and a processing device. The memory device stores instructions. The processing device is communicatively coupled to the memory device. The processing device is configured to execute the instructions to receive medical data pertaining to the patient. The processing device is also configured to execute the instructions to determine, by the artificial intelligence engine and by using one or more machine learning models, a disease progression level for the medical condition of the patient. The determining is based at least on the medical data. The disease progression level indicates a risk of the patient reaching a next stage on a disease continuum of the medical condition. The processing device is further configured to execute the instructions to generate, by the artificial intelligence engine, the treatment plan for the medical condition. The generating is based at least on the disease progression level. The treatment plan comprises one or more actionable items to be performed on or by the patient. The processing device is also configured to execute the instructions to transmit the treatment plan to a computing device for presentation to a healthcare professional.

The present disclosure further provides a tangible, non-transitory computer-readable medium storing instructions that, when executed, cause a processing device to receive medical data pertaining to a patient. The instructions also cause the processing device to determine, by an artificial intelligence engine and by using one or more machine learning models, a disease progression level for a medical condition of the patient. The determining is based at least on the medical data. The disease progression level indicates a risk of the patient reaching a next stage on a disease continuum of the medical condition. The instructions further cause the processing device to generate, by the artificial intelligence engine, a treatment plan for the medical condition. The generating is based at least on the disease progression level. The treatment plan comprises one or more actionable items to be performed on or by the patient. The instructions also cause the processing device to transmit the treatment plan to a computing device for presentation to a healthcare professional.

Other technical features may be readily apparent to one skilled in the art from the following figures, descriptions, and claims.

### BRIEF DESCRIPTION OF THE DRAWINGS

For a more complete understanding of the present disclosure and its advantages, reference is now made to the following description, taken in conjunction with the accompanying drawings. It is emphasized that, according to common practice, the various features of the drawings are not necessarily to-scale. On the contrary, the dimensions of the various features may be—and typically are—arbitrarily expanded or reduced for the purpose of clarity.

FIG. 1 is a block diagram of an example of a system for generating a treatment plan for a medical condition of a patient, in accordance with some implementations of the present disclosure.

FIG. 2 is a block diagram of an example of a computer system, in accordance with some implementations of the present disclosure.

FIG. 3 is a block diagram of an example of training a machine learning model to output, based on medical data pertaining to a patient, a disease progression level for a medical condition of the patient, in accordance with some implementations of the present disclosure.

FIG. 4 is a graph of an example of a patient encounter timeline, in accordance with some implementations of the present disclosure.

FIG. 5 is a graph of an example of a population encounter timeline, in accordance with some implementations of the present disclosure.

FIG. 6 is a graph of examples of risk values for a plurality of patients, in accordance with some implementations of the present disclosure.

FIG. 7 is a graph of an example of a plurality of patients divided into different risk groups based on their risk value, in accordance with some implementations of the present disclosure.

FIG. 8 is a flow diagram of an example of a method for generating a treatment plan for a medical condition of a patient, in accordance with some implementations of the present disclosure.

FIG. 9 is a diagram of an example of an overview display of a client portal presenting instances of gaps in treatment included in an instance of a treatment plan, in accordance with some implementations of the present disclosure.

FIG. 10 illustrates, in block diagram form, a system architecture that can be configured to provide a population health management service, in accordance with some implementations of the present disclosure.

FIG. 11 shows additional details of a knowledge cloud, in accordance with some implementations of the present disclosure.

FIG. 12 shows an example subject matter ontology, in accordance with some implementations of the present disclosure.

FIG. 13 shows aspects of a conversation, in accordance with some implementations of the present disclosure.

FIG. 14 shows a cognitive map or “knowledge graph”, in accordance with some implementations of the present disclosure.

### NOTATION AND NOMENCLATURE

Various terms are used to refer to particular system components. A particular component may be referred to commercially or otherwise by different names. Further, a particular component (or the same or similar component) may be referred to commercially or otherwise by different names. Consistent with this, nothing in the present disclosure shall be deemed to distinguish between components that differ only in name but not in function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example implementations only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example implementations. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” “inside,” “outside,” “contained within,” “superimposing upon,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

A “healthcare professional” may refer to a doctor, physician assistant, nurse, chiropractor, dentist, physical therapist, acupuncturist, physical trainer, coach, personal trainer, neurologist, cardiologist, or the like. A “healthcare professional” may also refer to any person with a credential, license, degree, or the like in the field of medicine, physical therapy, rehabilitation, or the like.

“Real-time” may refer to less than or equal to 2 seconds. “Near real-time” may refer to any interaction of a sufficiently short time to enable two individuals to engage in a dialogue via such user interface, and will generally be less than 10 seconds (or any suitable proximate difference between two different times) but greater than 2 seconds.

“Results” may refer to medical results or medical outcomes. Results and outcomes may refer to responses to medical actions. A “medical action(s)” may refer to any suitable action(s) performed by a healthcare professional, and such action or actions may include diagnoses, prescriptions for treatment plans, prescriptions for treatment apparatuses, and the making, composing and/or executing of appointments, telemedicine sessions, prescription of medicines, telephone calls, emails, text messages, and the like.

### DETAILED DESCRIPTION

The following discussion is directed to various implementations of the present disclosure. Although one or more of these implementations may be preferred, the implementations disclosed should not be interpreted, or otherwise used, as limiting the scope of the present disclosure, including the claims. In addition, one skilled in the art will understand that the following description has broad application, and the discussion of any implementation is meant only to be exemplary of that implementation, and not intended to intimate that the scope of the disclosure, including the claims, is limited to that implementation.

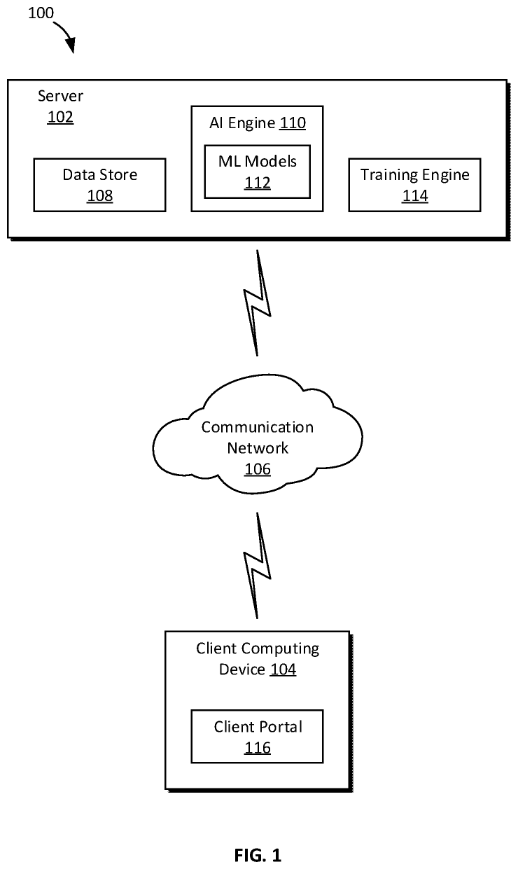

FIG. 1 is a block diagram of an example of a system 100 for generating treatment plans for a medical condition. The system 100 illustrated in FIG. 1 includes a server 102, a client computing device 104, and a communication network 106. The system 100 illustrated in FIG. 1 is provided as one example of such a system. The methods described herein may be used with systems with fewer, additional, or different components in different configurations than the system 100 illustrated in FIG. 1. For example, in some implementations, the system 100 may include additional computing devices, and may include additional servers.

The communication network 106 may be a wired network, a wireless network, or both. All or parts of the communication network 106 may be implemented using various networks, for example and without limitation, a cellular data network, the Internet, a Bluetooth™ network, a Near-Field Communications (NFC) network, a Z-Wave network, a ZigBee network, a wireless local area network (for example, Wi-Fi), a wireless accessory Personal Area Networks (PAN), cable, an Ethernet network, satellite, a machine-to-machine (M2M) autonomous network, and a public switched telephone network. Using suitable wireless or wired communication protocols, the various components of the system 100 may communicate with each other over the communication network 106. In some implementations, communications with other external devices (not shown) may occur over the communication network 106.

The server 102 is configured to store and to provide data related to managing treatment plans. The server 102 may include one or more computers and may take the form of a distributed and/or virtualized computer or computers. The server 102 may be configured to store data regarding treatment plans. For example, the server 102 may be configured to hold system data, such as data pertaining to treatment plans for treating one or more patients. The server 102 may also be configured to store data regarding performance by a patient in following a treatment plan. For example, the server 102 may be configured to hold medical data, such as data pertaining to one or more patients, including data representing each patient's performance within the treatment plan. In addition, the server 102 may store attributes (e.g., personal, performance, measurement, etc.) of patients, disease progression levels of medical conditions of patients, treatment plans followed by patients, and results of the treatment plans and may use correlations and other statistical or probabilistic measures to enable the partitioning of or to partition the disease progression levels into different patient cohort-equivalent databases. For example, the data for a first cohort of first patients having a first similar medical condition, a first similar disease progression level, a first treatment plan followed by the first patient, and a first result of the treatment plan may be stored in a first patient database. The data for a second cohort of second patients having a second similar medical condition, a second similar disease progression level, a second treatment plan followed by the second patient, and a second result of the treatment plan may be stored in a second patient database. Any single attribute or any combination of attributes may be used to separate the cohorts of patients. In some implementations, the different cohorts of patients may be stored in different partitions or volumes of the same database. There is no specific limit to the number of different cohorts of patients allowed, other than as limited by mathematical combinatoric and/or partition theory.

This attribute data, disease progression level data, treatment plan data, and results data may be obtained from and/or computing devices over time and stored, for example, in a data store 108. The attribute data, disease progression level data, treatment plan data, and results data may be correlated in patient-cohort databases. The attributes of the patients may include personal information, measurement information, healthcare encounters information, or a combination thereof.

In addition to historical information about other patients stored in the patient cohort-equivalent databases, real-time or near-real-time information based on the current patient's attribute about a current patient being treated may be stored in an appropriate patient cohort-equivalent database. The attribute of the patient may be determined to match or be similar to the attribute of another patient in a particular cohort (e.g., cohort A) and the patient may be assigned to that cohort.

Medical data may be stored in the data store 108 in the form of electronic health records (EHRs) that are associated with one or more patients. In some implementations, EHRs from different, disparate medical providers of a patient are stored in the data store 108. The health information exchanged between computing devices in the system 100 (e.g., between client computing device 104 and another computing device) may include health records associated with a patient such as medical and treatment histories of the patient but can go beyond standard clinical data collected by a healthcare provider. For example, health records may include a patient's medical history, diagnoses, medications, treatment plans, immunization dates, allergies, radiology images, and laboratory and test results.

In some implementations, the server 102 executes an AI engine (e.g., artificial intelligence engine 110) that uses one or more machine learning models 112 to perform at least one of the implementations disclosed herein. The server 102 may include a training engine 114 capable of generating the one or more machine learning models 112. The training engine 114 may be a rackmount server, a router computer, a personal computer, a portable digital assistant, a smartphone, a laptop computer, a tablet computer, a netbook, a desktop computer, an Internet of Things (IoT) device, any other desired computing device, or any combination of the above. The training engine 114 may be cloud-based, a real-time software platform, or an embedded system (e.g., microcode-based and/or implemented) and it may include privacy software or protocols, and/or security software or protocols.

The client computing device 104 may be used by a healthcare professional to obtain or provide information about patients. The client computing device 104 may also be used by the healthcare professional to obtain, monitor, and adjust treatment plans for patients. The client computing device 104 illustrates in FIG. 1 includes a client portal 116. The client portal 116 is configured to communicate information to a healthcare professional and to receive feedback from the healthcare professional. The client portal 116 may include one or more input devices (e.g., a keyboard, a mouse, a touch-screen input, a gesture sensor, a microphone, a processor configured for voice recognition, a telephone, a trackpad, or a combination thereof). The client portal 116 may also include one of more output devices (e.g., a computer monitor, a display screen on a tablet, smartphone, or a smart watch). The one or more output devices may include other hardware and/or software components such as a projector, virtual reality capability, augmented reality capability, etc. The one or more output devices may incorporate various different visual, audio, or other presentation technologies. For example, at least one of the output devices may include a non-visual display, such as an audio signal, which may include spoken language and/or other sounds such as tones, chimes, and/or melodies, which may signal different conditions and/or directions. At least one of the output devices may include one or more different display screens presenting various data and/or interfaces or controls for use by the user. At least one of the output devices may include graphics, which may be presented by a web-based interface and/or by a computer program or application (App.).

In some implementations, the client portal 116 may be configured to provide voice-based functionalities, with hardware and/or software configured to interpret spoken instructions by the healthcare professional by using one or more microphones. The client portal 116 may include functionality provided by or similar to existing voice-based assistants such as Siri by Apple, Alexa by Amazon, Google Assistant, or Bixby by Samsung. The client portal 116 may include other hardware and/or software components. The client portal 116 may include one or more general purpose devices and/or special-purpose devices.

In some implementations, the system 100 may provide computer translation of language to and/or from the client portal 116. The computer translation of language may include computer translation of spoken language and/or computer translation of text, wherein the text and/or spoken language may be any language, formal or informal, current or outdated, digital, quantum or analog, invented, human or animal (e.g., dolphin) or ancient, with respect to the foregoing, e.g., Old English, Zulu, French, Japanese, Klingon, Kobaian, Attic Greek, Modern Greek, etc., and in any form, e.g., academic, dialectical, patois, informal, e.g., “electronic texting,” etc. Additionally or alternatively, the system 100 may provide voice recognition and/or spoken pronunciation of text. For example, the system 100 may convert spoken words to printed text and/or the system 100 may audibly speak language from printed text. The system 100 may be configured to recognize spoken words by any or all of the patient and the healthcare professional. In some implementations, the system 100 may be configured to recognize and react to spoken requests or commands by the user. For example, the system 100 may automatically initiate a telemedicine session in response to a verbal command by a patient (which may be given in any one of several different languages).

In some implementations, the server 102 may generate aspects of the client portal 116 for presentation by the client portal 116. For example, the server 102 may include a web server configured to generate the display screens for presentation upon the client portal 116. For example, the artificial intelligence engine 110 may generate treatment plans for users and generate display screens including those treatment plans for presentation on the client portal 116. In some implementations, the client portal 116 may be configured to present a virtualized desktop hosted by the server 102. In some implementations, the server 102 may be configured to communicate with the client portal 116 via the communication network 106. In some implementations, the client portal 116 operates from a healthcare professional's location geographically separate from a location of the server 102.

In some implementations, the client portal 116 may be one of several different terminals (e.g., computing devices) that may be physically, virtually or electronically grouped together, for example, in one or more call centers or at one or more healthcare professionals' offices. In some implementations, multiple instances of the client portal 116 may be distributed geographically. In some implementations, a person may work as an assistant remotely from any conventional office infrastructure, including a home office. Such remote work may be performed, for example, where the client portal 116 takes the form of a computer and/or telephone. This remote work functionality may allow for work-from-home arrangements that may include full-time, part-time, and/or flexible work hours for an assistant.

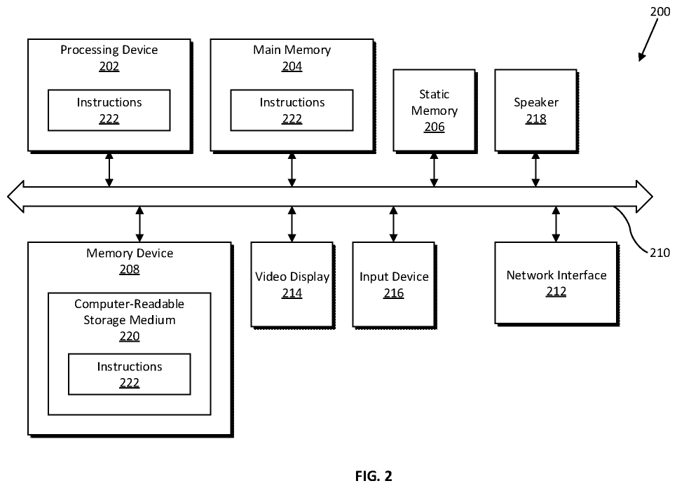

FIG. 2 is a block diagram of an example of a computer system 200 which can perform any one or more of the methods described herein, in accordance with one or more aspects of the present disclosure. In one example, the computer system 200 may include a computing device and correspond to one or more of the server 102 (including the artificial intelligence engine 110), the client computing device 104, or any suitable component of FIG. 1. The computer system 200 may be capable of executing instructions implementing the one or more machine learning models 112 of the artificial intelligence engine 110 of FIG. 1. The computer system 200 may be connected (e.g., networked) to other computer systems in a LAN, an intranet, an extranet, or the Internet, including via the cloud or a peer-to-peer network. The computer system 200 may operate in the capacity of a server in a client-server network environment. The computer system 200 may be a personal computer (PC), a tablet computer, a wearable (e.g., wristband), a set-top box (STB), a personal Digital Assistant (PDA), a mobile phone, a smartphone, a camera, a video camera, an Internet of Things (IoT) device, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 200 (one example of a “computing device”) illustrated in FIG. 2 includes a processing device 202, a main memory 204 (e.g., read-only memory (ROM), flash memory, solid state drives (SSDs), dynamic random access memory (DRAM) such as synchronous DRAM (SDRAM)), a static memory 206 (e.g., flash memory, solid state drives (SSDs), static random access memory (SRAM)), and a memory device 208, which communicate with each other via a bus 210.

The processing device 202 represents one or more general-purpose processing devices such as a microprocessor, central processing unit, or the like. More particularly, the processing device 202 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 202 may also be one or more special-purpose processing devices such as an application specific integrated circuit (ASIC), a system on a chip, a field programmable gate array (FPGA), a digital signal processor (DSP), network processor, or the like. The processing device 202 may be configured to execute instructions for performing any of the operations and steps discussed herein.

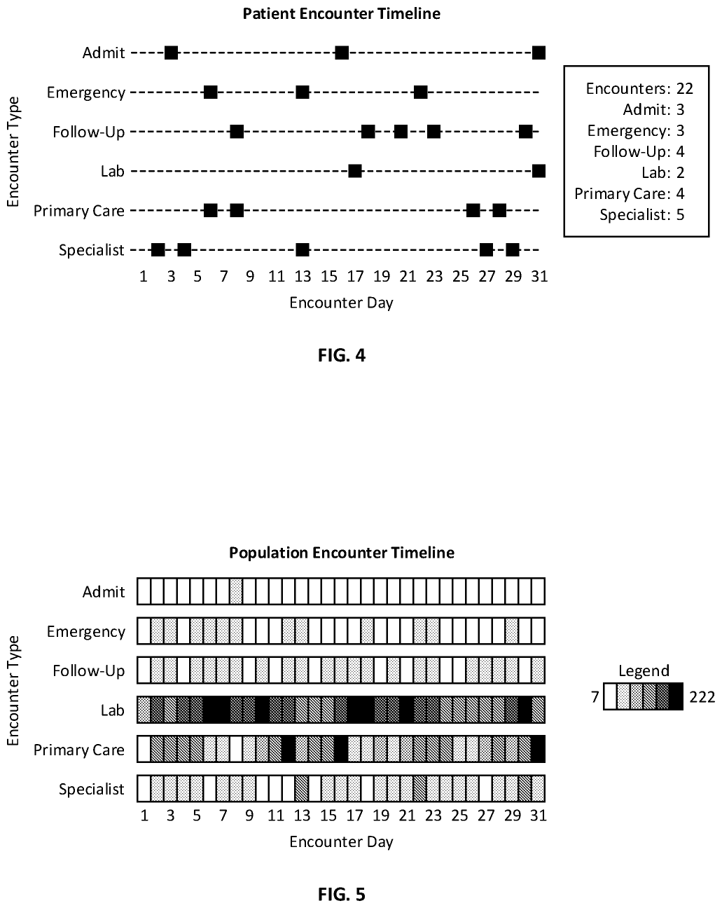

The computer system 200 illustrated in FIG. 4 further includes a network interface device 212. The computer system 200 also may include a video display 214 (e.g., a liquid crystal display (LCD), a light-emitting diode (LED), an organic light-emitting diode (OLED), a quantum LED, a cathode ray tube (CRT), a shadow mask CRT, an aperture grille CRT, a monochrome CRT), one or more input devices 216 (e.g., a keyboard and/or a mouse or a gaming-like control), and one or more speakers 218 (e.g., a speaker). In one illustrative example, the video display 214 and the input device(s) 216 may be combined into a single component or device (e.g., an LCD touch screen).

The memory device 208 may include a computer-readable storage medium 220 on which the instructions 222 embodying any one or more of the methods, operations, or functions described herein is stored. The instructions 222 may also reside, completely or at least partially, within the main memory 204 and/or within the processing device 202 during execution thereof by the computer system 200. As such, the main memory 204 and the processing device 202 also constitute computer-readable media. The instructions 222 may further be transmitted or received over a network via the network interface device 212.

While the computer-readable storage medium 220 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium capable of storing, encoding or carrying out a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

A medical condition may follow a disease continuum. For example, a medical condition may follow a disease continuum including stages of wellness, pre-disease, disease with no complications, disease with one complication, disease with multiple complications, palliative, and then deceased. Care pathways are evidence-based recommendations for treating a medical condition. For example, a care pathway may provide specific sets of evidence-based recommendations tailored to treat each stage of a medical condition. Patient outcome may be improved when evidence-based recommendations are further tailored to account for a disease progression level of a medical condition of a patient. The disease progression level indicates, among other things, a risk of a patient reaching the next stage on a disease continuum of a medical condition. For example, a first patient may be at a lower risk to reach the next stage on a disease continuum of a medical condition than a second patient. Thus, treatment recommendations that are effective at preventing the first patient from reaching the next stage of the disease continuum may be ineffective at preventing the second patient from reaching the next stage on the disease continuum. The disease progression level may also indicate a stage on a disease continuum of a medical condition that a patient is on.

Determine a patient's disease progression level may be a challenging problem. For example, a multitude of information may be considered when determining a patient's disease progression level, and such consideration may result in inaccuracies in the progression level selection process. The multitude of information considered may include, e.g., attributes of the patient such as personal information, measurement information, and healthcare encounters information. Personal information may include, e.g., demographic, psychographic or other information, such as an age, a gender, a medical condition, a familial medication history, an injury, a medical procedure, a medication prescribed, or any combination thereof. Measurement information may include, e.g., a weight, a height, a body mass index, a vital sign, a respiration rate, a heartrate, a temperature, a blood pressure, or any combination thereof. The healthcare encounters information may include statistics related to the patient's encounters with various healthcare professionals (e.g., hospital admissions, emergency room visits, follow-up visits, lab tests, primary care physician visits, specialist visits, or any combination thereof). Correlating a specific patient's attributes with known data for a cohort of other patients enables determination of the patient's disease progression level. Accounting for the patient's disease progression level enables generation of treatment plans that may result in preventing the patient from reaching the next stage on a disease continuum of a medical condition.

Accordingly, systems and methods, such as those described herein, that use artificial intelligence and/or machine learning to determine a disease progression level for a medical condition of a patient, may be desirable. For example, the machine learning models 112 may be trained to assign patients to certain cohorts based on their attributes, select disease progression levels using real-time and historical data correlations involving patient cohort-equivalents, and determine a treatment plan, among other things. The one or more machine learning models 112 may be generated by the training engine 114 and may be implemented in computer instructions executable by one or more processing devices of the training engine 114 and/or the server 102. To generate the one or more machine learning models 112, the training engine 114 may train the one or more machine learning models 112. The one or more machine learning models 112 may be used by the artificial intelligence engine 110.

To train the one or more machine learning models 112, the training engine 114 may use a training data set of a corpus of the attributes of other patients with the same medical condition, disease progression levels assigned to other patients, the treatment plans performed by the other patients, and the results of the other patients. The one or more machine learning models 112 may be trained to match patterns of attributes of a patient with attributes of other patients assigned to a particular cohort. The term “match” may refer to an exact match, or to correspondences, associations, relationships, approximations or other mathematical, linguistic and other non-exact matches, including, e.g., a correlative match, a substantial match, a partial match, an associative match, a relational match, etc. The one or more machine learning models 112 may be trained to receive the attributes of a patient as input, to map the attributes to attributes of other patients assigned to a cohort, and to select a disease progression level from that cohort.

Using training data that includes training inputs and corresponding target outputs, the one or more machine learning models 112 may refer to model artifacts created by the training engine 114. The training engine 114 may find patterns in the training data wherein such patterns map the training input to the target output, and generate the machine learning models 112 that capture these patterns. In some implementations, the artificial intelligence engine 110 and/or the training engine 114 may reside on another component (e.g., the client computing device 104) depicted in FIG. 1.

The one or more machine learning models 112 may comprise, e.g., a single level of linear or non-linear operations (e.g., a support vector machine [SVM]) or the machine learning models 112 may be a deep network, i.e., a machine learning model comprising multiple levels of non-linear operations. Examples of deep networks include neural networks, and neural networks may include generative adversarial networks, convolutional neural networks, recurrent neural networks with one or more hidden layers, and fully connected neural networks (e.g., wherein each neuron may transmit its output signal to the input of the remaining neurons, as well as to itself). For example, the machine learning model may include numerous layers and/or hidden layers that use various neurons to perform calculations (e.g., dot products).

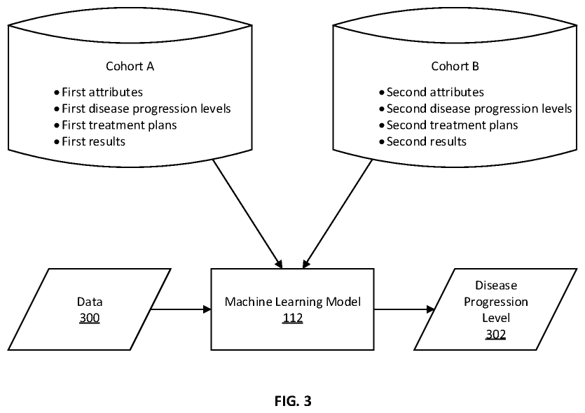

FIG. 3 is a block diagram of an example of training the machine learning model 112 to output, based on data 300 pertaining to the patient, a disease progression level 302 for a medical condition of the patient according to the present disclosure. Data pertaining to other patients may be received by the server 102. The data may include attributes of the other patients, the disease progression levels assigned to the other patients, the details of the treatment plans performed by the other patients, and/or the results of performing the treatment plans.

As depicted in FIG. 3, the data has been assigned to different cohorts. Cohort A includes data for patients having similar first attributes, first disease progression levels, first treatment plans, and first results. Cohort B includes data for patients having similar second attributes, second disease progression levels, second treatment plans, and second results. For example, cohort A may include first attributes of patients in their twenties without any additional medical conditions, and such cohort A patients' disease progression levels may indicate a low risk of reaching the next stage of a disease continuum. Further, cohort B may include second attributes of patients in their sixties with one or more additional medical conditions, and cohort B patients' disease progression levels may indicate a high risk of reaching the next stage of the disease continuum.

As further depicted in FIG. 3, cohort A and cohort B may be included in a training dataset used to train the machine learning model 112. The machine learning model 112 may be trained to match a pattern between one or more attributes for each cohort and to output a disease progression level 302 that provides the result, i.e., the best match. Accordingly, when the data 300 for a new patient is input into the trained machine learning model 112, the trained machine learning model 112 may match the one or more attributes included in the data 300 with one or more attributes in either cohort A or cohort B and output the appropriate disease progression level 302.

In some implementations, the artificial intelligence engine 110 determines a disease progression level for a patient based on medical encounters the patient has with one or more healthcare providers over a period of time. FIG. 4 is a graph of an example of a patient encounter timeline. The patient encounter timeline illustrated in FIG. 4 indicates the type and day of each encounter of a patient during a timeframe of 31 days. For example, the patient encounter timeline illustrated in FIG. 4 indicates the patient had an encounter with their primary care physician on day 26 and an encounter with a specialist on day 27. The graph illustrated in FIG. 4 is an example of a visual representation of patient encounters over the period of time that may be generated based on medical records from medical entities (or healthcare providers). This visual representation may be presented (e.g., to a healthcare professional) on a user interface (e.g., the client portal 116). Attributes related to medical encounters of the patient with one or more healthcare providers over a period of time may be used to determine the risk of the patient reaching the next stage on the disease continuum of a medical condition. Attributes related to medical encounters of the patient may include frequency-related attributes (i.e., how frequent certain types of encounters are happening). For example, a re-admission (i.e., a second admission at least 48 hours after a first admission) may be a frequency-related attribute. Further, a patient keeping on going back to their primary care physician or urgent care may be a frequency-related attribute. Alternatively, or in addition, attributes related to medical encounters of the patient may include intensity-related attributes (i.e., how many different types of encounters are happening). For example, a patient just using their primary care physician for lab tests (which is an example of a signature of a regular, well-managed diabetes patient) may be a low-intensity attribute. Further, a patient that has a lot of admissions, emergency encounters, follow-up encounters, and/or encounters with specialists may be a high-intensity attribute. Alternatively, or in addition, attributes related to medical encounters of the patient may include recency-related attributes (i.e., a cluster of recent encounters). For example, the patient encounter timelines illustrated in FIG. 4 includes three clusters (or episodes). Alternatively, or in addition, attributes related to medical encounters of the patient may include duration-related attributes (i.e., how long are a patient's episodes). For example, duration of plurality of clusters (e.g., a Lindsey cluster) may be a duration-related attribute. In some implementations, the artificial intelligence engine 110 may determine individual values for a plurality of patient encounter-related attributes, and then combine the individual values to determine a composite value (e.g., a risk value) that indicates a risk of the patient reaching the next stage on the disease continuum of a medical condition. For example, the artificial intelligence engine 110 may determine individual values for frequency-related attributes, intensity-related attributes, recency-related attributes, and duration-related attributes, and then combine the individual values to determine a risk value. In some implementations, when determining a composite value, the artificial intelligence engine 110 may apply the same (or different) weighting factors to each of the individual values. The weighting factors may be selected, e.g., based on the medical condition, one or more non-encounter related attributes of the patient, or both. In some implementation, the risk value is normalized to a predetermined range (e.g., a range between 1 and 100).

In some implementations, the artificial intelligence engine 110 determines the disease progression level for a patient by comparing the patient's encounter timeline to encounter timelines of a plurality of other patients. For example, the artificial intelligence engine 110 may compare the patient's encounter timeline to encounter timelines of a plurality of other patients with similar attributes (e.g., the same medical condition). FIG. 5 is a graph of an example of a population encounter timeline. The population encounter timeline illustrated in FIG. 5 indicates the number of patients for each type of encounter on each day during a timeframe of 31 days. The one or more machine learning models 112 may be trained using training data comprising a plurality of encounter timelines for a plurality of patients. For example, the training engine 114 may train one or more machine learning models 112 using the population encounter timeline illustrated in FIG. 5 as training data. By analyzing the population encounter timeline, the one or more machine learning models 112 identifies patterns that indicate different levels of risk of a patient reaching a next stage on a disease continuum of a medical condition. For example, the artificial intelligence engine 110 may determine a risk value for each patient in the population (e.g., using frequency-related attributes, intensity-related attributes, recency-related attributes, duration-related attributes, or a combination thereof).

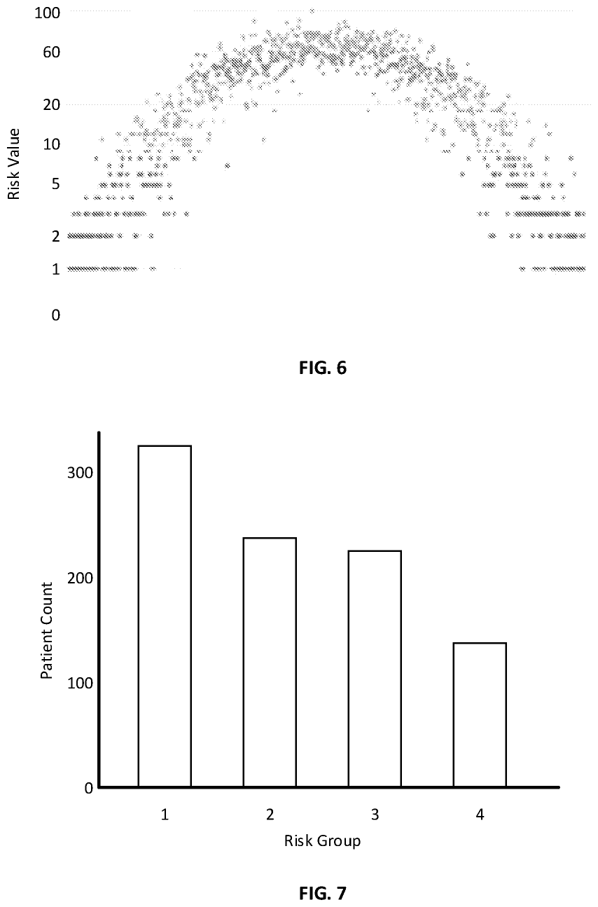

In some implementations, the artificial intelligence engine 110 stratifies the plurality of patients into different risk groups based on their risk values. For example, the artificial intelligence engine 110 may stratify the plurality of patients into four risk groups based on their risk values. FIG. 6 is a graph of example risk values for a plurality of patients. The risk values have been normalized to a scale of 0 to 100 and are plotted on a logarithmic scale. A plurality of patients may be split into several risk groups based on their normalized risk value. For example, the plurality of patients represented in FIG. 6 may be divided into four risk groups. FIG. 7 is a bar graph of an example of the population counts for the four risk groups. As illustrated in FIG. 7, the plurality of patient is stratified into the four risk groups such that risk group 1 (i.e., the risk group of patients with the least risk of reaching the next stage in the disease continuum) has the most patients and risk group 4 (the risk group of patients with the highest risk of reaching the next stage in the disease continuum) has the least patients.

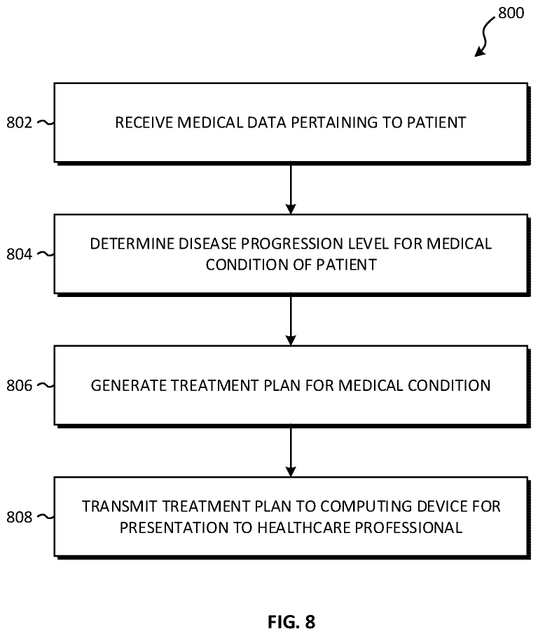

FIG. 8 is a flow diagram of an example of a method 800 for generating a treatment plan for a medical condition of a patient. The method 800 is performed by processing logic that may include hardware (circuitry, dedicated logic, etc.), software (such as is run on a general-purpose computer system, a dedicated machine, or a computing device of any kind (e.g., IoT node, wearable, smartphone, mobile device, etc.)), or a combination of both. The method 800 and/or each of its individual functions, routines, subroutines, or operations may be performed by one or more processors of a computing device (e.g., any component of FIG. 1, such as server 102 executing the artificial intelligence engine 110). In certain implementations, the method 800 may be performed by a single processing thread. Alternatively, the method 800 may be performed by two or more processing threads, wherein each thread implements one or more individual functions, routines, subroutines, or operations of the methods.

For simplicity of explanation, the method 800 is depicted in FIG. 8 and described as a series of operations performed by the artificial intelligence engine 110. However, operations in accordance with this disclosure can occur in various orders and/or concurrently, and/or with other operations not presented and described herein. For example, the operations depicted in the method 800 in FIG. 8 may occur in combination with any other operation of any other method disclosed herein. Furthermore, not all illustrated operations may be required to implement the method 800 in accordance with the disclosed subject matter. In addition, those skilled in the art will understand and appreciate that the method 800 could alternatively be represented via a state diagram or event diagram as a series of interrelated states.

At block 802, the artificial intelligence engine 110 receives medical data pertaining to the patient. In some implementations, the artificial intelligence engine 110 may receive the medical records from the data store 108, the client computing device 104, another computing device, a database, or a combination thereof. The medical data may include an encounter timeline for the patient that indicates medical encounters of the patient with one or more healthcare providers over a period of time. Alternatively or in addition, the medical data may include one or more treatment items that have been performed on the patient. Alternatively or in addition, the medical data may include any of or all the personal information, and/or measurement information previously described above.

At block 804, the artificial intelligence engine 110 determines a disease progression level for the medical condition of the patient using one or more machine learning models. For example, the artificial intelligence engine 110 determines the disease progression level based at least on the medical data using any of the methods described above.

At block 806, the artificial intelligence engine 110 generates a treatment plan for the medical condition. The artificial intelligence engine 110 generates the treatment plan based at least on the disease progression level. The treatment plan includes one or more actionable items to be performed on or by the patient. Actionable items may include changes to medications prescribed to the patient. For example, the treatment plan may indicate the patient should start taking one or more new medications, adjust dosage levels of one or more medications, stop taking one or more medications, or a combination thereof. Alternatively, or in addition, actionable items may include one or more lab test to perform on the patient. For example, the treatment plan may indicate the patient should start having one or more new lab tests, adjust the frequency of one or more lab tests, stop having one or more lab tests, or a combination thereof. Alternatively, or in addition, actionable items may include one or more items related to patient compliance. For example, the treatment plan may indicate the patient is not having labs taken at prescribed intervals, the patient is not taking medication as prescribed, the patient is not visiting their primary care physician at prescribed intervals, or a combination thereof. Alternatively, or in addition, actionable items may include one or more healthcare attributes of the patient to monitor. For example, the treatment plan may indicate that the healthcare professional should start monitoring the patient's creatinine level. Alternatively, or in addition, actionable items may include one or more recommendations for specialists to evaluate the patient. For example, the treatment plan may include a recommendation for an eye doctor to evaluate a patient experiencing blind spots. In some implementations, the artificial intelligence engine 110 generates the treatment plan by determining a plurality of recommended treatment items for the patient based at least on the disease progression level and comparing the plurality of recommended treatment items with a plurality of performed treatment items (indicated, e.g., in the medical data pertaining to the patient received at block 802) to determine the one or more actionable items.

Given the often limited amount of a time allotted for analyzing a patient's medical history, it can be challenging for a healthcare professional to identify all potential gaps in treatments. Thus, in some implementations, actionable items may include gaps in treatment for the patient. Gaps in treatment for the patient may include items related to patient compliance. For example, the treatment plan may indicate the patient is not having labs taken at prescribed intervals, the patient is not taking medication as prescribed, the patient is not visiting their primary care physician at prescribed intervals, or a combination thereof. Alternatively, or in addition, gaps in treatment for the patient may include evidence-based recommendations (e.g., included in a care pathway) that are not being followed. For example, the artificial intelligence engine 110 may determine that the medical data pertaining to the patient received at block 802 indicates one or more evidence-based recommendations are not being followed. Alternatively, or in addition, gaps in treatment for the patient may include changes based on the disease progression level determined at block 804. For example, the artificial intelligence engine 110 may determine a plurality of recommended treatment items for the patient based at least on the disease progression level. Then, the artificial intelligence engine 110 may determine one or more actionable items to include in the treatment plan by comparing the plurality of recommended treatment items with a plurality of performed treatment items indicated, for example, in the medical data pertaining to the patient received at block 802.

At block 808, the treatment plan is transmitted to a computing device for presentation to a healthcare professional. For example, the server 102 may transmit the treatment plan to the client computing device 104, another computing device, or a combination thereof. In some implementations, the treatment plan may be presented to the healthcare professional on a user interface as a visual communication, a tactile communication, an acoustic communication, or a combination thereof. For example, the client portal 116 may display text and/or image(s) indicating treatment plans, gaps in treatment, actionable items, other items, or a combination thereof. Alternatively, or in addition, the client portal 116 may emit audible instructions indicating treatment plans, gaps in treatment, actionable items, other items, or a combination thereof.

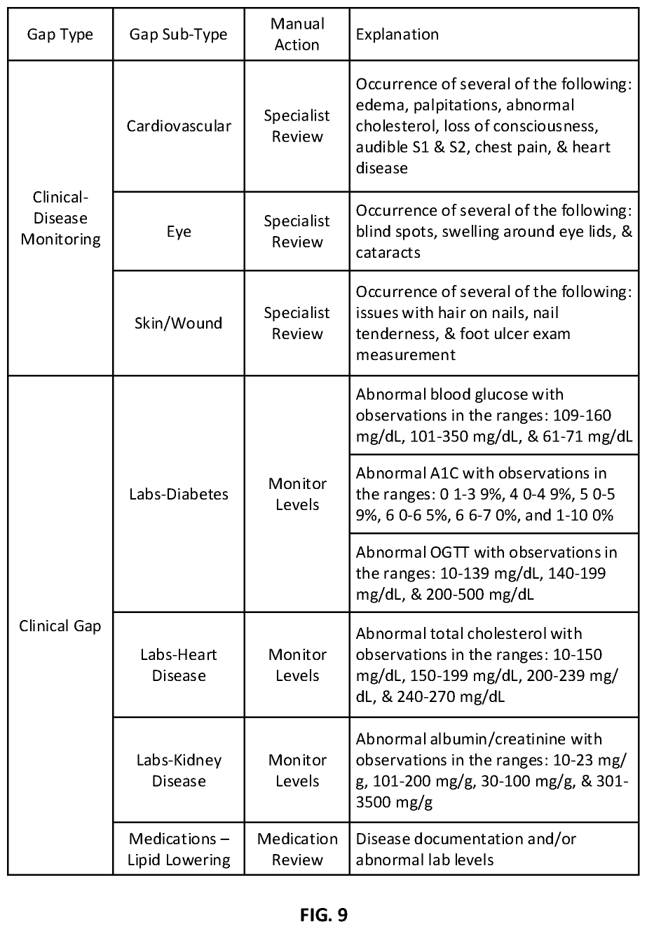

FIG. 9 is a diagram of an example of an overview display 900 of the client portal 116 presenting instances of gaps in treatment included in an instance of a treatment plan. The overview display 900 illustrated in FIG. 9 includes text indicating the type and sub-type of each gap in care. The text in FIG. 9 also indicates a suggested manual action to take for each gap in care. Further, the text in FIG. 9 indicates an explanation of why the gap in treatment was detected. Given that treatment plans are often approved by a healthcare professional, including an explanation for each gap in treatment may make it easy for a healthcare professional to determine how each gap in treatment should be handled. There may be valid reasons from some gaps in treatment. For example, extended gaps between a patient having labs taken may be due insurance requirements. Further, a patient may have contraindications to a specific medication.

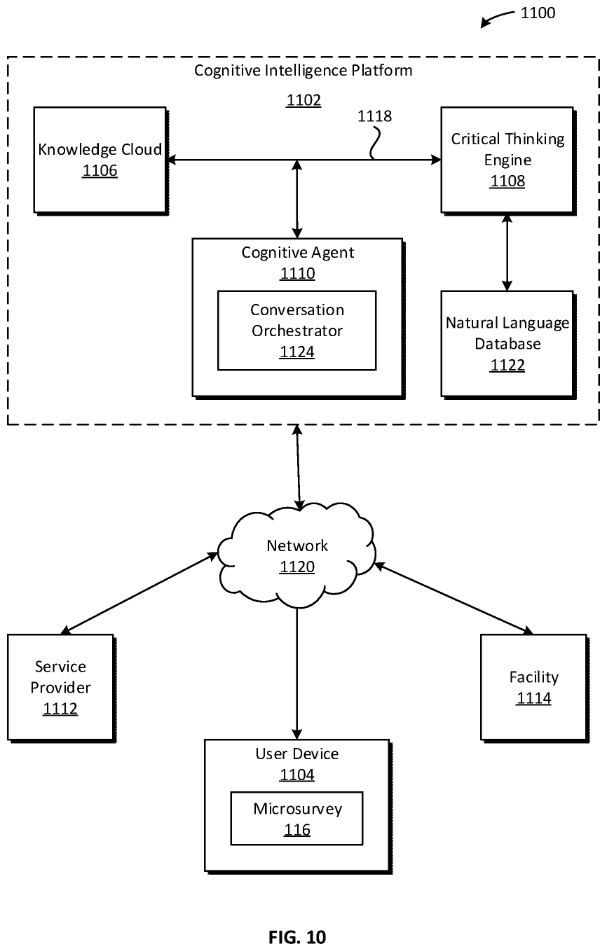

FIG. 10 shows a system architecture 1100 that can be configured to provide a population health management service, in accordance with various implementations. Specifically, FIG. 10 illustrates a high-level overview of an overall architecture that includes a cognitive intelligence platform 1102 communicably coupled to a user device 1104. In some implementations, the cognitive intelligence platform 1102 performs any or all of the functions the server 102 and/or the artificial intelligence engine 110 illustrated in FIG. 1 and described above. The cognitive intelligence platform 1102 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing the cognitive intelligence platform 1102 can represent any form of computing device without departing from the scope of the present disclosure.

The several computing devices work in conjunction to implement components of the cognitive intelligence platform 1102 including: a knowledge cloud 1106; a critical thinking engine 1108; a natural language database 1122; and a cognitive agent 1110. The cognitive intelligence platform 1102 is not limited to implementing only these components, or in the manner described in FIG. 10. That is, other system architectures can be implemented, with different or additional components, without departing from the scope of the present disclosure. The example system architecture 1100 illustrates one way to implement the methods and techniques described herein.

The knowledge cloud 1106 represents a set of instructions executing within the cognitive intelligence platform 1102 that implement a database configured to receive inputs from several sources and entities. For example, some of the sources an entities include a service provider 1112, a facility 1114, and a microsurvey 1116—each described further below.

The critical thinking engine 1108 represents a set of instructions executing within the cognitive intelligence platform 1102 that execute tasks using artificial intelligence, such as recognizing and interpreting natural language (e.g., performing conversational analysis), and making decisions in a linear manner (e.g., in a manner similar to how the human left brain processes information). Specifically, an ability of the cognitive intelligence platform 1102 to understand natural language is powered by the critical thinking engine 1108. In various implementations, the critical thinking engine 1108 includes a natural language database 1122. The natural language database 1122 includes data curated over at least thirty years by linguists and computer data scientists, including data related to speech patterns, speech equivalents, and algorithms directed to parsing sentence structure.

Furthermore, the critical thinking engine 1108 is configured to deduce causal relationships given a particular set of data, where the critical thinking engine 1108 is capable of taking the individual data in the particular set, arranging the individual data in a logical order, deducing a causal relationship between each of the data, and drawing a conclusion. The ability to deduce a causal relationship and draw a conclusion (referred to herein as a “causal” analysis) is in direct contrast to other implementations of artificial intelligence that mimic the human left brain processes. For example, the other implementations can take the individual data and analyze the data to deduce properties of the data or statistics associated with the data (referred to herein as an “analytical” analysis). However, these other implementations are unable to perform a causal analysis—that is, deduce a causal relationship and draw a conclusion from the particular set of data. As described further below—the critical thinking engine 1108 is capable of performing both types of analysis: causal and analytical.

The cognitive agent 1110 represents a set of instructions executing within the cognitive intelligence platform 1102 that implement a client-facing component of the cognitive intelligence platform 1102. The cognitive agent 1110 is an interface between the cognitive intelligence platform 1102 and the user device 1104. And in some implementations, the cognitive agent 1110 includes a conversation orchestrator 1124 that determines pieces of communication that are presented to the user device 1104 (and the user). When a user of the user device 1104 interacts with the cognitive intelligence platform 1102, the user interacts with the cognitive agent 1110. The several references herein, to the cognitive agent 1110 performing a method, can implicate actions performed by the critical thinking engine 1108, which accesses data in the knowledge cloud 1106 and the natural language database 1122.

In various implementations, the several computing devices executing within the cognitive intelligence platform are communicably coupled by way of a network/bus interface. Furthermore, the various components (e.g., the knowledge cloud 1106, the critical thinking engine 1108, and the cognitive agent 1110), are communicably coupled by one or more inter-host communication protocols 1118. In one example, the knowledge cloud 1106 is implemented using a first computing device, the critical thinking engine 1108 is implemented using a second computing device, and the cognitive agent 1110 is implemented using a third computing device, where each of the computing devices are coupled by way of the inter-host communication protocols 1118. Although in this example, the individual components are described as executing on separate computing devices this example is not meant to be limiting, the components can be implemented on the same computing device, or partially on the same computing device, without departing from the scope of the present disclosure.

The user device 1104 represents any form of a computing device, or network of computing devices, e.g., a personal computing device, a smart phone, a tablet, a wearable computing device, a notebook computer, a media player device, and a desktop computing device. The user device 1104 includes a processor, at least one memory, and at least one storage. A user uses the user device 1104 to input a given text posed in natural language (e.g., typed on a physical keyboard, spoken into a microphone, typed on a touch screen, or combinations thereof) and interacts with the cognitive intelligence platform 1102, by way of the cognitive agent 1110.

The system architecture 1100 includes a network 1120 that communicatively couples various devices, including the cognitive intelligence platform 1102 and the user device 1104. The network 1120 can include local area network (LAN) and wide area networks (WAN). The network 1120 can include wired technologies (e.g., Ethernet®) and wireless technologies (e.g., Wi-Fi®, code division multiple access (CDMA), global system for mobile (GSM), universal mobile telephone service (UMTS), Bluetooth®, and ZigBee®. For example, the user device 1104 can use a wired connection or a wireless technology (e.g., Wi-Fi®) to transmit and receive data over the network 1120.

Still referring to FIG. 10, the knowledge cloud 1106 is configured to receive data from various sources and entities and integrate the data in a database. An example source that provides data to the knowledge could 1106 is the service provider 1112, an entity that provides a type of service to a user. For example, the service provider 1112 can be a health service provider (e.g., a doctor's office, a physical therapist's office, a nurse's office, or a clinical social worker's office), and a financial service provider (e.g., an accountant's office). For purposes of this discussion, the cognitive intelligence platform 1102 provides services in the health industry, thus the examples discussed herein are associated with the health industry. However, any service industry can benefit from the disclosure herein, and thus the examples associated with the health industry are not meant to be limiting.

Throughout the course of a relationship between the service provider 1112 and a user (e.g., the service provider 1112 provides healthcare to a patient), the service provider 1112 collects and generates data associated with the patient or the user, including health records that include doctor's notes and prescriptions, billing records, and insurance records. The service provider 1112, using a computing device (e.g., a desktop computer or a tablet), provides the data associated with the user to the cognitive intelligence platform 1102, and more specifically the knowledge cloud 1106.

Another example source that provides data to the knowledge cloud 1106 is the facility 1114. The facility 1114 represents a location owned, operated, or associated with any entity including the service provider 1112. As used herein, an entity represents an individual or a collective with a distinct and independent existence. An entity can be legally recognized (e.g., a sole proprietorship, a partnership, a corporation) or less formally recognized in a community. For example, the entity can include a company that owns or operates a gym (facility). Additional examples of the facility 1114 include, but is not limited to, a hospital, a trauma center, a clinic, a dentist's office, a pharmacy, a store (including brick and mortar stores and online retailers), an out-patient care center, a specialized care center, a birthing center, a gym, a cafeteria, and a psychiatric care center.

As the facility 1114 represents a large number of types of locations, for purposes of this discussion and to orient the reader by way of example, the facility 1114 represents the doctor's office or a gym. The facility 1114 generates additional data associated with the user such as appointment times, an attendance record (e.g., how often the user goes to the gym), a medical record, a billing record, a purchase record, an order history, and an insurance record. The facility 1114, using a computing device (e.g., a desktop computer or a tablet), provides the data associated with the user to the cognitive intelligence platform 1102, and more specifically the knowledge cloud 1106.

An additional example source that provides data to the knowledge cloud 1106 is the microsurvey 1116. The microsurvey 1116 represents a tool created by the cognitive intelligence platform 1102 that enables the knowledge cloud 1106 to collect additional data associated with the user. The microsurvey 1116 is originally provided by the cognitive intelligence platform 1102 (by way of the cognitive agent 1110) and the user provides data responsive to the microsurvey 1116 using the user device 1104. Additional details of the microsurvey 1116 are described below.

Yet another example source that provides data to the knowledge cloud 1106, is the cognitive intelligence platform 1102, itself. In order to address the care needs and well-being of the user, the cognitive intelligence platform 1102 collects, analyzes, and processes information from the user, healthcare providers, and other eco-system participants, and consolidates and integrates the information into knowledge. The knowledge can be shared with the user and stored in the knowledge cloud 1106.

In various implementations, the computing devices used by the service provider 1112 and the facility 1114 are communicatively coupled to the cognitive intelligence platform 1102, by way of the network 1120. While data is used individually by various entities including: a hospital, practice group, facility, or provider, the data is less frequently integrated and seamlessly shared between the various entities in the current art. The cognitive intelligence platform 1102 provides a solution that integrates data from the various entities. That is, the cognitive intelligence platform 1102 ingests, processes, and disseminates data and knowledge in an accessible fashion, where the reason for a particular answer or dissemination of data is accessible by a user.

In particular, the cognitive intelligence platform 1102 (e.g., by way of the cognitive agent 1110 interacting with the user) holistically manages and executes a health plan for durational care and wellness of the user (e.g., a patient or consumer). The health plan includes various aspects of durational management that is coordinated through a care continuum.

The cognitive agent 1110 can implement various personas that are customizable. For example, the personas can include knowledgeable (sage), advocate (coach), and witty friend (jester). And in various implementations, the cognitive agent 1110 persists with a user across various interactions (e.g., conversations streams), instead of being transactional or transient. Thus, the cognitive agent 1110 engages in dynamic conversations with the user, where the cognitive intelligence platform 1102 continuously deciphers topics that a user wants to talk about. The cognitive intelligence platform 1102 has relevant conversations with the user by ascertaining topics of interest from a given text posed in a natural language input by the user. Additionally the cognitive agent 1110 connects the user to healthcare service providers, hyperlocal health communities, and a variety of services and tools/devices, based on an assessed interest of the user.

As the cognitive agent 1110 persists with the user, the cognitive agent 1110 can also act as a coach and advocate while delivering pieces of information to the user based on tonal knowledge, human-like empathies, and motivational dialog within a respective conversational stream, where the conversational stream is a technical discussion focused on a specific topic. Overall, in response to a question—e.g., posed by the user in natural language—the cognitive intelligence platform 1102 consumes data from and related to the user and computes an answer. The answer is generated using a rationale that makes use of common sense knowledge, domain knowledge, evidence-based medicine guidelines, clinical ontologies, and curated medical advice. Thus, the content displayed by the cognitive intelligence platform 1102 (by way of the cognitive agent 1110) is customized based on the language used to communicate with the user, as well as factors such as a tone, goal, and depth of topic to be discussed.

Overall, the cognitive intelligence platform 1102 is accessible to a user, a hospital system, and physician. Additionally, the cognitive intelligence platform 1102 is accessible to paying entities interested in user behavior—e.g., the outcome of physician-consumer interactions in the context of disease or the progress of risk management. Additionally, entities that provides specialized services such as tests, therapies, and clinical processes that need risk based interactions can also receive filtered leads from the cognitive intelligence platform 1102 for potential clients.

Conversational Analysis

In various implementations, the cognitive intelligence platform 1102 is configured to perform conversational analysis in a general setting. The topics covered in the general setting is driven by the combination of agents (e.g., cognitive agent 1110) selected by a user. In some implementations, the cognitive intelligence platform 1102 uses conversational analysis to identify the intent of the user (e.g., find data, ask a question, search for facts, find references, and find products) and a respective micro-theory in which the intent is logical.

For example, the cognitive intelligence platform 1102 applies conversational analysis to decode what the user is asking or stated, where the question or statement is in free form language (e.g., natural language). Prior to determining and sharing knowledge (e.g., with the user or the knowledge cloud 1106), using conversational analysis, the cognitive intelligence platform 1102 identifies an intent of the user and overall conversational focus.

The cognitive intelligence platform 1102 responds to a statement or question according to the conversational focus and steers away from another detected conversational focus so as to focus on a goal defined by the cognitive agent 1110. Given an example statement of a user, “I want to fly out tomorrow,” the cognitive intelligence platform 1102 uses conversational analysis to determine an intent of the statement. Is the user aspiring to be bird-like or does he want to travel? In the former case, the micro-theory is that of human emotions whereas in the latter case, the micro-theory is the world of travel. Answers are provided to the statement depending on the micro-theory in which the intent logically falls.

The cognitive intelligence platform 1102 utilize a combination of linguistics, artificial intelligence, and decision trees to decode what a user is asking or stating. The discussion includes methods and system design considerations and results from an existing implementation. Additional details related to conversational analysis are discussed next.

Analyzing Conversational Context as Part of Conversational Analysis

For purposes of this discussion, the concept of analyzing conversational context as part of conversational analysis is now described. To analyze conversational context, the following steps are taken: 1) obtain text (e.g., receive a question) and perform translations; 2) understand concepts, entities, intents, and micro-theory; 3) relate and search; 4) ascertain the existence of related concepts; 5) logically frame concepts or needs; 6) understand the questions that can be answered from available data; and 7) answer the question. Each of the foregoing steps is discussed next, in turn.

Step 1: Obtain Text/Question and Perform Translations

In various implementations, the cognitive intelligence platform 1102 (FIG. 10) receives a text or question and performs translations as appropriate. The cognitive intelligence platform 1102 supports various methods of input including text received from a touch interface (e.g., options presented in a microsurvey), text input through a microphone (e.g., words spoken into the user device), and text typed on a keyboard or on a graphical user interface. Additionally, the cognitive intelligence platform 1102 supports multiple languages and auto translation (e.g., from English to Traditional/Simplified Chinese or vice versa).

The example text below is used to described methods in accordance with various implementations herein:
“One day in January 1913. G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

The cognitive intelligence platform 1102 analyzes the example text above to detect structural elements within the example text (e.g., paragraphs, sentences, and phrases). In some implementations, the example text is compared to other sources of text such as dictionaries, and other general fact databases (e.g., Wikipedia) to detect synonyms and common phrases present within the example text.

Step 2: Understand Concept, Entity, Intent, and Micro-Theory

In step 2, the cognitive intelligence platform 1102 parses the text to ascertain concepts, entities, intents, and micro-theories. An example output after the cognitive intelligence platform 1102 initially parses the text is shown below, where concepts, and entities are shown in bold.
“One day in January 1913. G. H. Hardy, a famous Cambridge University mathematician received a letter from an Indian named Srinivasa Ramanujan asking him for his opinion of 120 mathematical [symbol] that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the [symbol] laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

For example, the cognitive intelligence platform 1102 ascertains that Cambridge is a university—which is a full understanding of the concept. The cognitive intelligence platform (e.g., the cognitive agent 1110) understands what humans do in Cambridge, and an example is described below in which the cognitive intelligence platform 1102 performs steps to understand a concept.

For example, in the context of the above example, the cognitive agent 1110 understands the following concepts and relationships:
Cambridge employed John Edensor Littlewood (1)
Cambridge has the position Ramanujan's position at Cambridge University (2)
Cambridge employed G. H. Hardy. (3)

The cognitive agent 1110 also assimilates other understandings to enhance the concepts, such as:
Cambridge has Trinity College as a suborganization. (4)
Cambride is located in Cambridge. (5)
Alan Turing is previously enrolled at Cambridge. (6)
Stephen Hawking attended Cambridge. (7)

The statements (1)-(7) are not picked at random. Instead the cognitive agent 1110 dynamically constructs the statements (1)-(7) from logic or logical inferences based on the example text above. Formally, the example statements (1)-(7) are captured as follows:
(#$subOrganizations #$UniversityOfCambridge #$TrinityCollege-Cambridge-England) (8)
(#$placeInCity #$UniversityOfCambridge #$Cityof CambridgeEngland) (9)
(#$schooling #$AlanTuring #$UniversityOfCambridge #$PreviouslyEnrolled) (10)
(#$hasAlumni #$UniversityOfCambridge #$StephenHawking) (11)

Step 3: Relate and Search

Next, in step 3, the cognitive agent 1110 relates various entities and topics and follows the progression of topics in the example text. Relating includes the cognitive agent 1110 understanding the different instances of Hardy are all the same person, and the instances of Hardy are different from the instances of Littlewood. The cognitive agent 1110 also understands that the instances Hardy and Littlewood share some similarities—e.g., both are mathematicians and they did some work together at Cambridge on Number Theory. The ability to track this across the example text is referred to as following the topic progression with a context.

Step 4: Ascertain the Existence of Related Concepts

Next, in Step 4, the cognitive agent 1110 asserts non-existent concepts or relations to form new knowledge. Step 4 is an optional step for analyzing conversational context. Step 4 enhances the degree to which relationships are understood or different parts of the example text are understood together. If two concepts appear to be separate—e.g., a relationship cannot be graphically drawn or logically expressed between enough sets of concepts—there is a barrier to understanding. The barriers are overcome by expressing additional relationships. The additional relationships can be discovered using strategies like adding common sense or general knowledge sources (e.g., using the common sense data 1208) or adding in other sources including a lexical variant database, a dictionary, and a thesaurus.

One example of concept progression from the example text is as follows: the cognitive agent 1110 ascertains the phrase “theorems that Ramanujan said he had discovered” is related to the phrase “his results”, which is related to “Ramanujan's work is in number theory, a branch of mathematics that deals with the subtle laws and relationships that govern numbers.”

Step 5: Logically Frame Concepts or Needs

In Step 5, the cognitive agent 1110 determines missing parameters—which can include for example, missing entities, missing elements, and missing nodes—in the logical framework (e.g., with a respective micro-theory). The cognitive agent 1110 determines sources of data that can inform the missing parameters. Step 5 can also include the cognitive agent 1110 adding common sense reasoning and finding logical paths to solutions.

With regards to the example text, some common sense concepts include:
Mathematicians develop Theorems. (12)
Theorems are hard to comprehend. (13)
Interpretations are not apparent for years. (14)
Applications are developed over time. (15)
Mathematicians collaborate and assess work. (16)

With regards to the example text, some passage concepts include:
Ramanujan did Theorems in Early 20^{th} Century. (17)
Hardy assessed Ramanujan's Theorems. (18)
Hardy collaborated with Littlewood. (19)
Hardy and Littlewood assessed Ramanujan's work (20)

Within the micro-theory of the passage analysis, the cognitive agent 1110 understands and catalogs available paths to answer questions. In Step 5, the cognitive agent 1110 makes the case that the concepts (12)-(20) are expressed together.

Step 6: Understand the Questions that can be Answered from Available Data

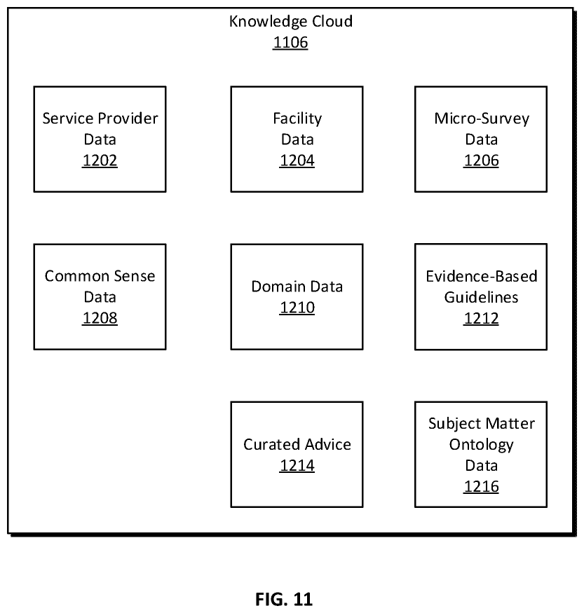

In Step 6, the cognitive agent 1110 parses sub-intents and entities. Given the example text, the following questions are answerable from the cognitive agent's developed understanding of the example text, where the understanding was developed using information and context ascertained from the example text as well as the common sense data 1208 (FIG. 11):
What situation causally contributed to Ramanujan's position at Cambridge? (21)
Does the author of the passage regret that Ramanujan died prematurely? (22)
Does the author of the passage believe that Ramanujan is a mathematical genius? (23)

Based on the information that is understood by the cognitive agent 1110, the questions (21)-(23) can be answered.

By using an exploration method such as random walks, the cognitive agent 1110 makes a determination as the paths that are plausible and reachable with the context (e.g., micro-theory) of the example text. Upon explorations, the cognitive agent 1110 catalogs a set of meaningful questions. The set of meaningful questions are not asked, but instead explored based on the cognitive agent's understanding of the example text.

Given the example text, an example of exploration that yields a positive result is: “a situation X that caused Ramanujan's position.” In contrast, an example of exploration that causes irrelevant results is: “a situation Y that caused Cambridge.” The cognitive agent 1110 is able to deduce that the latter exploration is meaningless, in the context of a micro-theory, because situations do not cause universities. Thus the cognitive agent 1110 is able to deduce, there are no answers to Y, but there are answers to X.

Step 7: Answer the Question

In Step 7, the cognitive agent 1110 provides a precise answer to a question. For an example question such as: “What situation causally contributed to Ramanujan's position at Cambridge?” the cognitive agent 1110 generates a precise answer using the example reasoning:
HardyandLittlewoodsEvaluatingOfRamanujansWork (24)
HardyBeliefThatRamanujanIsAnExpertInMathematics (25)
HardysBeliefThatRamanujanIsAnExpertInMathematicsAndAGenius (26)

In order to generate the above reasoning statements (24)-(26), the cognitive agent 1110 utilizes a solver or prover in the context of the example text's micro-theory—and associated facts, logical entities, relations, and assertions. As an additional example, the cognitive agent 1110 uses a reasoning library that is optimized for drawing the example conclusions above within the fact, knowledge, and inference space (e.g., work space) that the cognitive agent 1110 maintains.

By implementing the steps 1-7, the cognitive agent 1110 analyzes conversational context. The described method for analyzing conversation context can also be used for recommending items in conversations streams. A conversational stream is defined herein as a technical discussion focused on specific topics. As related to described examples herein, the specific topics relate to health (e.g., diabetes). Throughout the lifetime of a conversational stream, a cognitive agent 1110 collect information over may channels such as chat, voice, specialized applications, web browsers, contact centers, and the like.

By implementing the methods to analyze conversational context, the cognitive agent 1110 can recommend a variety of topics and items throughout the lifetime of the conversational stream. Examples of items that can be recommended by the cognitive agent 1110 include: surveys, topics of interest, local events, devices or gadgets, dynamically adapted health assessments, nutritional tips, reminders from a health events calendar, and the like.

Accordingly, the cognitive intelligence platform 1102 provides a platform that codifies and takes into consideration a set of allowed actions and a set of desired outcomes. The cognitive intelligence platform 1102 relates actions, the sequences of subsequent actions (and reactions), desired sub-outcomes, and outcomes, in a way that is transparent and logical (e.g., explainable). The cognitive intelligence platform 1102 can plot a next best action sequence and a planning basis (e.g., health care plan template, or a financial goal achievement template), also in a manner that is explainable. The cognitive intelligence platform 1102 can utilize a critical thinking engine 1108 and a natural language database 1122 (e.g., a linguistics and natural language understanding system) to relate conversation material to actions.

For purposes of this discussion, several examples are discussed in which conversational analysis is applied within the field of durational and whole-health management for a user. The discussed implementations holistically address the care needs and well-being of the user during the course of his life. The methods and systems described herein can also be used in fields outside of whole-health management, including: phone companies that benefits from a cognitive agent; hospital systems or physicians groups that want to coach and educate patients; entities interested in user behavior and the outcome of physician-consumer interactions in terms of a progress of disease or risk management; entities that provide specialized services (e.g., test, therapies, clinical processes) to filter leads; and sellers, merchants, stores and big box retailers that want to understand which product to sell.

FIG. 11 shows additional details of a knowledge cloud, in accordance with various implementations. In particular, FIG. 11 illustrates various types of data received from various sources, including service provider data 1202, facility data 1204, microsurvey data 1206, common sense data 1208, domain data 1210, evidence-based guidelines 1212, subject matter ontology data 1214, and curated advice 1216. The types of data represented by the service provider data 1202 and the facility data 1204 include any type of data generated by the service provider 1112 and the facility 1114, and the above examples are not meant to be limiting. Thus, the example types of data are not meant to be limiting and other types of data can also be stored within the knowledge cloud 1106 without departing from the scope of the present disclosure.

The service provider data 1202 is data provided by the service provider 1112 (described in FIG. 10) and the facility data 1204 is data provided by the facility 1114 (described in FIG. 10). For example, the service provider data 1202 includes medical records of a respective patient of a service provider 1112 that is a doctor. In another example, the facility data 1204 includes an attendance record of the respective patient, where the facility 1114 is a gym. The microsurvey data 1206 is data provided by the user device 1104 responsive to questions presented in the microsurvey 1116 (FIG. 10).

Common sense data 1208 is data that has been identified as “common sense”, and can include rules that govern a respective concept and used as glue to understand other concepts.

Domain data 1210 is data that is specific to a certain domain or subject area. The source of the domain data 1210 can include digital libraries. In the healthcare industry, for example, the domain data 1210 can include data specific to the various specialties within healthcare such as, obstetrics, anesthesiology, and dermatology, to name a few examples. In the example described herein, the evidence-based guidelines 1212 include systematically developed statements to assist practitioner and patient decisions about appropriate health care for specific clinical circumstances.

Curated advice 1216 includes advice from experts in a subject matter. The curated advice 1216 can include peer-reviewed subject matter, and expert opinions. Subject matter ontology data 1214 includes a set of concepts and categories in a subject matter or domain, where the set of concepts and categories capture properties and relationships between the concepts and categories.

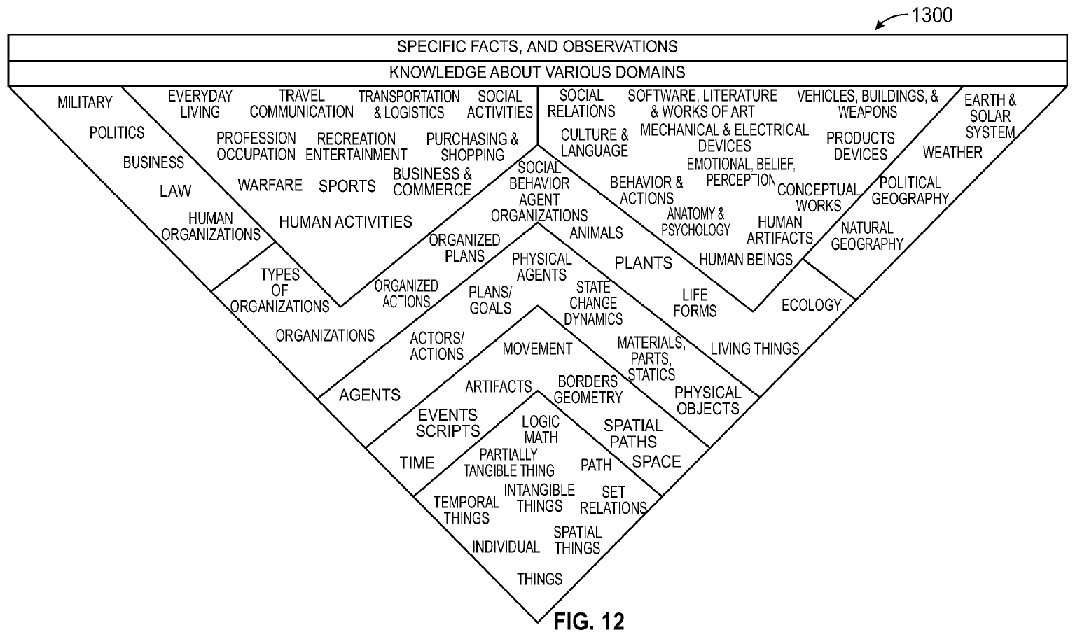

In particular, FIG. 12 illustrates an example subject matter ontology 1300 that is included as part of the subject matter ontology data 1214.

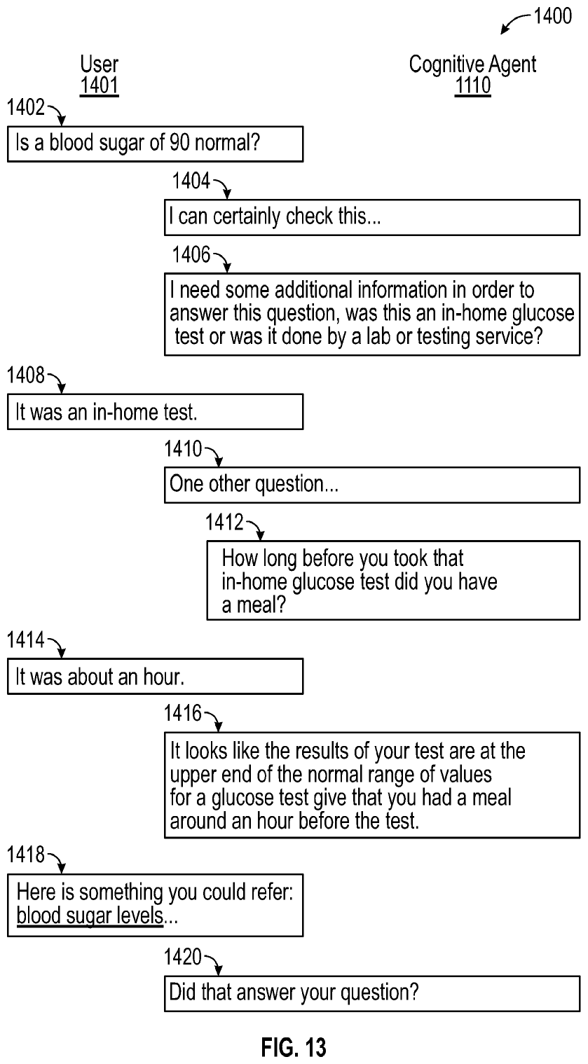

FIG. 13 illustrates aspects of a conversation 1400 between a user and the cognitive intelligence platform 1102, and more specifically the cognitive agent 1110. For purposes of this discussion, the user 1401 is a patient of the service provider 1112. The user interacts with the cognitive agent 1110 using a computing device, a smart phone, or any other device configured to communicate with the cognitive agent 1110 (e.g., the user device 1104 in FIG. 10). The user can enter text into the device using any known means of input including a keyboard, a touchscreen, and a microphone. The conversation 1400 represents an example graphical user interface (GUI) presented to the user 1401 on a screen of his computing device.

Initially, the user asks a general question, which is treated by the cognitive agent 1110 as an “originating question.” The originating question is classified into any number of potential questions (“pursuable questions”) that are pursued during the course of a subsequent conversation. In some implementations, the pursuable questions are identified based on a subject matter domain or goal. In some implementations, classification techniques are used to analyze language (e.g., such as those outlined in HPS ID20180901-01_method for conversational analysis). Any known text classification technique can be used to analyze language and the originating question. For example, in line 1402, the user enters an originating question about a subject matter (e.g., blood sugar) such as: “Is a blood sugar of 90 normal”?

In response to receiving an originating question, the cognitive intelligence platform 1102 (e.g., the cognitive agent 1110 operating in conjunction with the critical thinking engine 1108) performs a first round of analysis (e.g., which includes conversational analysis) of the originating question and, in response to the first round of analysis, creates a workspace and determines a first set of follow up questions.

In various implementations, the cognitive agent 1110 may go through several rounds of analysis executing within the workspace, where a round of analysis includes: identifying parameters, retrieving answers, and consolidating the answers. The created workspace can represent a space where the cognitive agent 1110 gathers data and information during the processes of answering the originating question. In various implementations, each originating question corresponds to a respective workspace. The conversation orchestrator 1124 can assess data present within the workspace and query the cognitive agent 1110 to determine if additional data or analysis should be performed.

In particular, the first round of analysis is performed at different levels, including analyzing natural language of the text, and analyzing what specifically is being asked about the subject matter (e.g., analyzing conversational context). The first round of analysis is not based solely on a subject matter category within which the originating question is classified. For example, the cognitive intelligence platform 1102 does not simply retrieve a predefined list of questions in response to a question that falls within a particular subject matter, e.g., blood sugar. That is, the cognitive intelligence platform 1102 does not provide the same list of questions for all questions related to the particular subject matter. Instead, for example, the cognitive intelligence platform 1102 creates dynamically formulated questions, curated based on the first round of analysis of the originating question.

In particular, during the first round of analysis, the cognitive agent 1110 parses aspects of the originating question into associated parameters. The parameters represent variables useful for answering the originating question. For example, the question “is a blood sugar of 90 normal” may be parsed and associated parameters may include, an age of the inquirer, the source of the value 90 (e.g., in home test or a clinical test), a weight of the inquirer, and a digestive state of the user when the test was taken (e.g., fasting or recently eaten). The parameters identify possible variables that can impact, inform, or direct an answer to the originating question.

For purposes of the example illustrated in FIG. 13, in the first round of analysis, the cognitive intelligence platform 1102 inserts each parameter into the workspace associated with the originating question (line 1402). Additionally, based on the identified parameters, the cognitive intelligence platform 1102 identifies a customized set of follow up questions (“a first set of follow-up questions). The cognitive intelligence platform 1102 inserts first set of follow-up questions in the workspace associated with the originating question.

The follow up questions are based on the identified parameters, which in turn are based on the specifics of the originating question (e.g., related to an identified micro-theory). Thus the first set of follow-up questions identified in response to, if a blood sugar is normal, will be different from a second set of follow up questions identified in response to a question about how to maintain a steady blood sugar.

After identifying the first set of follow up questions, in this example first round of analysis, the cognitive intelligence platform 1102 determines which follow up question can be answered using available data and which follow-up question to present to the user. As described over the next few paragraphs, eventually, the first set of follow-up questions is reduced to a subset (“a second set of follow-up questions”) that includes the follow-up questions to present to the user.

In various implementations, available data is sourced from various locations, including a user account, the knowledge cloud 1106, and other sources. Other sources can include a service that supplies identifying information of the user, where the information can include demographics or other characteristics of the user (e.g., a medical condition, a lifestyle). For example, the service can include a doctor's office or a physical therapist's office.

Another example of available data includes the user account. For example, the cognitive intelligence platform 1102 determines if the user asking the originating question, is identified. A user can be identified if the user is logged into an account associated with the cognitive intelligence platform 1102. User information from the account is a source of available data. The available data is inserted into the workspace of the cognitive agent 1110 as a first data.

Another example of available data includes the data stored within the knowledge cloud 1106. For example, the available data includes the service provider data 1202 (FIG. 11), the facility data 1204, the microsurvey data 1206, the common sense data 1208, the domain data 1210, the evidence-based guidelines 1212, the curated advice 1214, and the subject matter ontology data 1214. Additionally data stored within the knowledge cloud 1106 includes data generated by the cognitive intelligence platform 1102, itself.

Follow up questions presented to the user (the second set of follow-up questions) are asked using natural language and are specifically formulated (“dynamically formulated question”) to elicit a response that will inform or fulfill an identified parameter. Each dynamically formulated question can target one parameter at a time. When answers are received from the user in response to a dynamically formulated question, the cognitive intelligence platform 1102 inserts the answer into the workspace. In some implementations, each of the answers received from the user and in response to a dynamically formulated question, is stored in a list of facts. Thus the list of facts include information specifically received from the user, and the list of facts is referred to herein as the second data.

With regards to the second set of follow-up questions (or any set of follow-up questions), the cognitive intelligence platform 1102 calculates a relevance index, where the relevance index provides a ranking of the questions in the second set of follow-up questions. The ranking provides values indicative of how relevant a respective follow-up question is to the originating question. To calculate the relevance index, the cognitive intelligence platform 1102 can use conversations analysis techniques described in HPS ID20180901-01_method. In some implementations, the first set or second set of follow up questions is presented to the user in the form of the microsurvey 1116.

In this first round of analysis, the cognitive intelligence platform 1102 consolidates the first and second data in the workspace and determines if additional parameters need to be identified, or if sufficient information is present in the workspace to answer the originating question. In some implementations, the cognitive agent 1110 (FIG. 10) assesses the data in the workspace and queries the cognitive agent 1110 to determine if the cognitive agent 1110 needs more data in order to answer the originating question. The conversation orchestrator 1124 executes as an interface.

For a complex originating question, the cognitive intelligence platform 1102 can go through several rounds of analysis. For example, in a first round of analysis the cognitive intelligence platform 1102 parses the originating question. In a subsequent round of analysis, the cognitive intelligence platform 1102 can create a sub question, which is subsequently parsed into parameters in the subsequent round of analysis. The cognitive intelligence platform 1102 is smart enough to figure out when all information is present to answer an originating question without explicitly programming or pre-programming the sequence of parameters that need to be asked about.

In some implementations, the cognitive agent 1110 is configured to process two or more conflicting pieces of information or streams of logic. That is, the cognitive agent 1110, for a given originating question can create a first chain of logic and a second chain of logic that leads to different answers. The cognitive agent 1110 has the capability to assess each chain of logic and provide only one answer. That is, the cognitive agent 1110 has the ability to process conflicting information received during a round of analysis.

Additionally, at any given time, the cognitive agent 1110 has the ability to share its reasoning (chain of logic) to the user. If the user does not agree with an aspect of the reasoning, the user can provide that feedback which results in affecting change in a way the critical thinking engine 1108 analyzed future questions and problems.

Subsequent to determining enough information is present in the workspace to answer the originating question, the cognitive agent 1110 answers the question, and additionally can suggest a recommendation or a recommendation (e.g., line 1418). The cognitive agent 1110 suggests the reference or the recommendation based on the context and questions being discussed in the conversation (e.g., conversation 1400). The reference or recommendation serves as additional handout material to the user and is provided for informational purposes. The reference or recommendation often educates the user about the overall topic related to the originating question.

In the example illustrated in FIG. 13, in response to receiving the originating questions (line 1402), the cognitive intelligence platform 1102 (e.g., the cognitive agent 1110 in conjunction with the critical thinking engine 1108) parses the originating question to determine at least one parameter: location. The cognitive intelligence platform 1102 categorizes this parameter, and a corresponding dynamically formulated question in the second set of follow-up questions. Accordingly, in lines 1404 and 1406, the cognitive agent 1110 responds by notifying the user “I can certainly check this . . . ” and asking the dynamically formulated question “I need some additional information in order to answer this question, was this an in-home glucose test or was it done by a lab or testing service?”

The user 1401 enters his answer in line 1408: “It was an in-home test,” which the cognitive agent 1110 further analyzes to determine additional parameters: e.g., a digestive state, where the additional parameter and a corresponding dynamically formulated question as an additional second set of follow-up questions. Accordingly, the cognitive agent 1110 poses the additional dynamically formulated question in lines 1410 and 1412: “One other question . . . ” and “How long before you took that in-home glucose test did you have a meal?” The user provides additional information in response “it was about an hour” (line 1414).

The cognitive agent 1110 consolidates all the received responses using the critical thinking engine 1108 and the knowledge cloud 1106 and determines an answer to the initial question posed in line 1402 and proceeds to follow up with a final question to verify the user's initial question was answered. For example, in line 1416, the cognitive agent 1110 responds: “It looks like the results of your test are at the upper end of the normal range of values for a glucose test given that you had a meal around an hour before the test.” The cognitive agent 1110 provides additional information (e.g., provided as a link): “Here is something you could refer,” (line 1418), and follows up with a question “Did that answer your question?” (line 1420).

As described above, due to the natural language database 1122, in various implementations, the cognitive agent 1110 is able to analyze and respond to questions and statements made by a user 1401 in natural language. That is, the user 1401 is not restricted to using certain phrases in order for the cognitive agent 1110 to understand what a user 1401 is saying. Any phrasing, similar to how the user would speak naturally can be input by the user and the cognitive agent 1110 has the ability to understand the user.

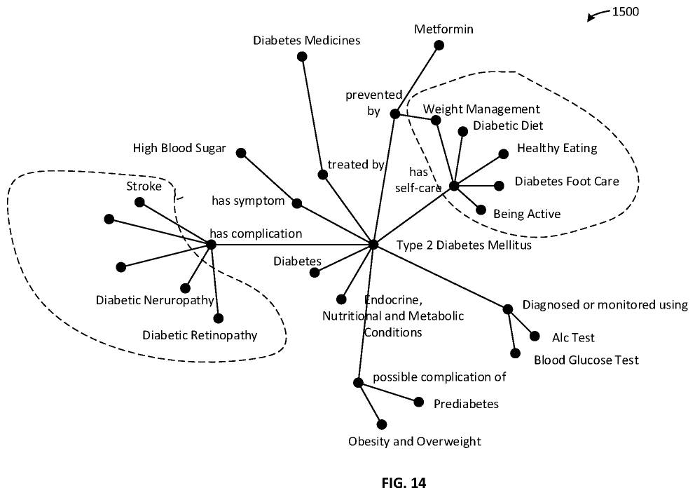

FIG. 14 illustrates a cognitive map or “knowledge graph” 1500, in accordance with various implementations. In particular, the knowledge graph represents a graph traversed by the cognitive intelligence platform 1102, when assessing questions from a user with Type 2 diabetes. Individual nodes in the knowledge graph 1500 represent a health artifact or relationship that is gleaned from direct interrogation or indirect interactions with the user (by way of the user device 1104).

In one implementations, the cognitive intelligence platform 1102 identified parameters for an originating question based on a knowledge graph illustrated in FIG. 14. For example, the cognitive intelligence platform 1102 parses the originating question to determine which parameters are present for the originating question. In some implementations, the cognitive intelligence platform 1102 infers the logical structure of the parameters by traversing the knowledge graph 1500, and additionally, knowing the logical structure enables the cognitive agent 1110 to formulate an explanation as to why the cognitive agent 1110 is asking a particular dynamically formulated question.

Consistent with the above disclosure, the examples of systems and methods enumerated in the following clauses are specifically contemplated and are intended as a non-limiting set of examples.

Clause 1. A method for generating, by an artificial intelligence engine, a treatment plan for a medical condition of a patient, the method comprising:
receiving medical data pertaining to the patient;
determining, by the artificial intelligence engine and by using one or more machine learning models, a disease progression level for the medical condition of the patient, wherein the determining is based at least on the medical data, and wherein the disease progression level indicates a risk of the patient reaching a next stage on a disease continuum of the medical condition;
generating, by the artificial intelligence engine, the treatment plan for the medical condition, wherein the generating is based at least on the disease progression level, and wherein the treatment plan comprises one or more actionable items to be performed on or by the patient; and
transmitting the treatment plan to a computing device for presentation to a healthcare professional.

Clause 2. The method of any clause herein, wherein the medical data pertaining to the patient comprises an encounter timeline for the patient that indicates medical encounters of the patient with one or more healthcare providers over a period of time, wherein determining the disease progression level further comprising determining the risk of the patient reaching the next stage on the disease continuum of the medical condition based at least on one or more attributes related to the medical encounters of the patient.

Clause 3. The method of any clause herein, wherein the one or more attributes related to the medical encounters of the patient comprise at least one selected from the group consisting of frequency, intensity, recency, and duration.

Clause 4. The method of any clause herein, further comprises training, by the artificial intelligence engine, the one or more machine learning models with training data comprising at least a plurality of encounter timelines for a plurality of other patients.

Clause 5. The method of any clause herein, wherein the one or more actionable items comprise gaps in treatment for the patient.

Clause 6. The method of any clause herein, wherein the medical data pertaining to the patient comprises a plurality of performed treatment items, wherein generating the treatment plan further comprises:
determining a plurality of recommended treatment items for the patient based at least on the disease progression level; and
comparing the plurality of recommended treatment items with the plurality of performed treatment items to determine the one or more actionable items.

Clause 7. The method of any clause herein, wherein the treatment plan further comprises explanations for each of the one or more actionable items, wherein the method further comprises displaying, on a user interface, the one or more actionable items and the explanations for each of the one or more actionable items.

Claim 8. A system for generating, by an artificial intelligence engine, a treatment plan for a medical condition of a patient, the system comprising:
a memory device for storing instructions; and
a processing device communicatively coupled to the memory device, the processing device configured to execute the instructions to:
   receive medical data pertaining to the patient,
   determine, by the artificial intelligence engine and by using one or more machine learning models, a disease progression level for the medical condition of the patient, wherein the determining is based at least on the medical data, and wherein the disease progression level indicates a risk of the patient reaching a next stage on a disease continuum of the medical condition,
   generate, by the artificial intelligence engine, the treatment plan for the medical condition, wherein the generating is based at least on the disease progression level, and wherein the treatment plan comprises one or more actionable items to be performed on or by the patient, and
   transmit the treatment plan to a computing device for presentation to a healthcare professional.

Clause 9. The system of any clause herein, wherein the medical data pertaining to the patient comprises an encounter timeline for the patient that indicates medical encounters of the patient with one or more healthcare providers over a period of time, wherein, to determine the disease progression level, the processing device is further configured to execute the instructions to determine the risk of the patient reaching the next stage on the disease continuum of the medical condition based at least on one or more attributes related to the medical encounters of the patient.

Clause 10. The system of any clause herein, wherein the one or more attributes related to the medical encounters of the patient comprise at least one selected from the group consisting of frequency, intensity, recency, and duration.

Clause 11. The system of any clause herein, wherein the processing device is further configured to execute the instructions to train, by the artificial intelligence engine, the one or more machine learning models with training data comprising at least a plurality of encounter timelines for a plurality of other patients.

Clause 12. The system of any clause herein, wherein the one or more actionable items comprise gaps in treatment for the patient.

Clause 13. The system of any clause herein, wherein the medical data pertaining to the patient comprises a plurality of performed treatment items, wherein, to generate the treatment plan, the processing device is further configured to execute the instructions to:
determine a plurality of recommended treatment items for the patient based at least on the disease progression level, and
compare the plurality of recommended treatment items with the plurality of performed treatment items to determine the one or more actionable items.

Clause 14. The system of any clause herein, wherein the treatment plan further comprises explanations for each of the one or more actionable items, wherein the system further comprises a user interface configured to display the one or more actionable items and the explanations for each of the one or more actionable items.

Clause 15. A tangible, non-transitory computer-readable medium storing instructions that, when executed, cause a processing device to:
receive medical data pertaining to a patient;
determine, by an artificial intelligence engine and by using one or more machine learning models, a disease progression level for a medical condition of the patient, wherein the determining is based at least on the medical data, and wherein the disease progression level indicates a risk of the patient reaching a next stage on a disease continuum of the medical condition;
generate, by the artificial intelligence engine, a treatment plan for the medical condition, wherein the generating is based at least on the disease progression level, and wherein the treatment plan comprises one or more actionable items to be performed on or by the patient; and
transmit the treatment plan to a computing device for presentation to a healthcare professional.

Clause 16. The computer-readable medium of any clause herein, wherein the medical data pertaining to the patient comprises an encounter timeline for the patient that indicates medical encounters of the patient with one or more healthcare providers over a period of time, wherein, to determine the disease progression level, the instructions further cause the processing device to determine the risk of the patient reaching the next stage on the disease continuum of the medical condition based at least on one or more attributes related to the medical encounters of the patient.

Clause 17. The computer-readable medium of any clause herein, wherein the one or more attributes related to the medical encounters of the patient comprise at least one selected from the group consisting of frequency, intensity, recency, and duration.

Clause 18. The computer-readable medium of any clause herein, wherein the instructions further cause the processing device to train, by the artificial intelligence engine, the one or more machine learning models with training data comprising at least a plurality of encounter timelines for a plurality of other patients.

Clause 19. The computer-readable medium of any clause herein, wherein the medical data pertaining to the patient comprises a plurality of performed treatment items, wherein, to determine the disease progression level, the instructions further cause the processing device to:
determine a plurality of recommended treatment items for the patient based on the disease progression level, and
compare the plurality of recommended treatment items with the plurality of performed treatment items to determine the one or more actionable items.

Clause 20. The computer-readable medium of any clause herein, wherein the treatment plan further comprises explanations for each of the one or more actionable items to be presented to the healthcare professional.

No part of the description in this application should be read as implying that any particular element, step, or function is an essential element that must be included in the claim scope. The scope of patented subject matter is defined only by the claims. Moreover, none of the claims is intended to invoke 25 U.S.C. § 104(f) unless the exact words “means for” are followed by a participle.

The foregoing description, for purposes of explanation, use specific nomenclature to provide a thorough understanding of the described embodiments. However, it should be apparent to one skilled in the art that the specific details are not required to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present invention. Once the above disclosure is fully appreciated, numerous variations and modifications will become apparent to those skilled in the art. It is intended that the following claims be interpreted to embrace all such variations and modifications.

What is claimed is:

1. A method for generating, by an artificial intelligence engine, a treatment plan for a medical condition of a patient, the method comprising:
receiving medical data pertaining to the patient;
determining, by the artificial intelligence engine and by using one or more machine learning models, a disease progression level for the medical condition of the patient, wherein the determining is based at least on the medical data, and wherein the disease progression level indicates a risk of the patient reaching a next stage on a disease continuum of the medical condition;
generating, by the artificial intelligence engine, the treatment plan for the medical condition, wherein the generating is based at least on the disease progression level, and wherein the treatment plan comprises one or more actionable items to be performed on or by the patient; and
transmitting the treatment plan to a computing device for presentation to a healthcare professional.

2. The method of claim 1, wherein the medical data pertaining to the patient comprises an encounter timeline for the patient that indicates medical encounters of the patient with one or more healthcare providers over a period of time, wherein determining the disease progression level further comprising determining the risk of the patient reaching the next stage on the disease continuum of the medical condition based at least on one or more attributes related to the medical encounters of the patient.

3. The method of claim 2, wherein the one or more attributes related to the medical encounters of the patient comprise at least one selected from the group consisting of frequency, intensity, recency, and duration.

4. The method of claim 2, further comprises training, by the artificial intelligence engine, the one or more machine learning models with training data comprising at least a plurality of encounter timelines for a plurality of other patients.

5. The method of claim 1, wherein the one or more actionable items comprise gaps in treatment for the patient.

6. The method of claim 5, wherein the medical data pertaining to the patient comprises a plurality of performed treatment items, wherein generating the treatment plan further comprises:
determining a plurality of recommended treatment items for the patient based at least on the disease progression level; and
comparing the plurality of recommended treatment items with the plurality of performed treatment items to determine the one or more actionable items.

7. The method of claim 1, wherein the treatment plan further comprises explanations for each of the one or more actionable items, wherein the method further comprises displaying, on a user interface, the one or more actionable items and the explanations for each of the one or more actionable items.

8. A system for generating, by an artificial intelligence engine, a treatment plan for a medical condition of a patient, the system comprising:
a memory device for storing instructions; and
a processing device communicatively coupled to the memory device, the processing device configured to execute the instructions to:
receive medical data pertaining to the patient,
determine, by the artificial intelligence engine and by using one or more machine learning models, a disease progression level for the medical condition of the patient, wherein the determining is based at least on the medical data, and wherein the disease progression level indicates a risk of the patient reaching a next stage on a disease continuum of the medical condition,
generate, by the artificial intelligence engine, the treatment plan for the medical condition, wherein the generating is based at least on the disease progression level, and wherein the treatment plan comprises one or more actionable items to be performed on or by the patient, and
transmit the treatment plan to a computing device for presentation to a healthcare professional.

9. The system of claim 8, wherein the medical data pertaining to the patient comprises an encounter timeline for the patient that indicates medical encounters of the patient with one or more healthcare providers over a period of time, wherein, to determine the disease progression level, the processing device is further configured to execute the instructions to determine the risk of the patient reaching the next stage on the disease continuum of the medical condition based at least on one or more attributes related to the medical encounters of the patient.

10. The system of claim 9, wherein the one or more attributes related to the medical encounters of the patient comprise at least one selected from the group consisting of frequency, intensity, recency, and duration.

11. The system of claim 9, wherein the processing device is further configured to execute the instructions to train, by the artificial intelligence engine, the one or more machine learning models with training data comprising at least a plurality of encounter timelines for a plurality of other patients.

12. The system of claim 8, wherein the one or more actionable items comprise gaps in treatment for the patient.

13. The system of claim 12, wherein the medical data pertaining to the patient comprises a plurality of performed treatment items, wherein, to generate the treatment plan, the processing device is further configured to execute the instructions to:
determine a plurality of recommended treatment items for the patient based at least on the disease progression level, and
compare the plurality of recommended treatment items with the plurality of performed treatment items to determine the one or more actionable items.

14. The system of claim 8, wherein the treatment plan further comprises explanations for each of the one or more actionable items, wherein the system further comprises a user interface configured to display the one or more actionable items and the explanations for each of the one or more actionable items.

15. A tangible, non-transitory computer-readable medium storing instructions that, when executed, cause a processing device to:
receive medical data pertaining to a patient;
determine, by an artificial intelligence engine and by using one or more machine learning models, a disease progression level for a medical condition of the patient, wherein the determining is based at least on the medical data, and wherein the disease progression level indicates a risk of the patient reaching a next stage on a disease continuum of the medical condition;
generate, by the artificial intelligence engine, a treatment plan for the medical condition, wherein the generating is based at least on the disease progression level, and wherein the treatment plan comprises one or more actionable items to be performed on or by the patient; and
transmit the treatment plan to a computing device for presentation to a healthcare professional.

16. The computer-readable medium of claim 15, wherein the medical data pertaining to the patient comprises an encounter timeline for the patient that indicates medical encounters of the patient with one or more healthcare providers over a period of time, wherein, to determine the disease progression level, the instructions further cause the processing device to determine the risk of the patient reaching the next stage on the disease continuum of the medical condition based at least on one or more attributes related to the medical encounters of the patient.

17. The computer-readable medium of claim 16, wherein the one or more attributes related to the medical encounters of the patient comprise at least one selected from the group consisting of frequency, intensity, recency, and duration.

18. The computer-readable medium of claim 16, wherein the instructions further cause the processing device to train, by the artificial intelligence engine, the one or more machine learning models with training data comprising at least a plurality of encounter timelines for a plurality of other patients.

19. The computer-readable medium of claim 15, wherein the medical data pertaining to the patient comprises a plurality of performed treatment items, wherein, to determine the disease progression level, the instructions further cause the processing device to:
determine a plurality of recommended treatment items for the patient based on the disease progression level, and
compare the plurality of recommended treatment items with the plurality of performed treatment items to determine the one or more actionable items.

20. The computer-readable medium of claim 15, wherein the treatment plan further comprises explanations for each of the one or more actionable items to be presented to the healthcare professional.
